(19) United States
(12) Patent Application Publication
(10) Pub. No.: US 2026/0216683 A1
(43) Pub. Date: Jul. 30, 2026

(54) COMPARTMENTALIZED ARRAYS, METHODS OF PREPARING AND USES THEREOF

(71) Applicant: University of Guelph, Guelph (CA)

(72) Inventors: Huiyan Li, Guelph (CA); Roshan Aggarwal, Rockwood (CA)

(73) Assignee: University of Guelph, Guelph, ON (CA)

(21) Appl. No.: 19/457,982

(22) Filed: Jan. 23, 2026

Related U.S. Application Data
(60) Provisional application No. 63/750,452, filed on Jan. 28, 2025.

(51) Int. Cl. B01J 19/00; G01N 33/543

(52) U.S. Cl. CPC B01J 19/0046; G01N 33/54393; B01J 2219/0061; B01J 2219/00637; B01J 2219/00664

(57) ABSTRACT

The present application describes compartmentalized arrays, methods of preparing and uses thereof for multiplexed bioanalysis.

### RELATED APPLICATIONS

The present application claims the benefit of priority from U.S. provisional patent application Ser. No. 63/750,452 filed on Jan. 28, 2025, the entire contents of which are incorporated herein by reference in their entirety.

### FIELD

The present application relates to compartmentalized arrays, methods of preparing and uses thereof.

### BACKGROUND

Multiplex biosensors are valuable tools for detecting and quantifying analytes in biomedical research, clinical diagnostics, and pharmaceutical development. For example, complex diseases such as cancer or cardiovascular disease can alter the expression levels of hundreds of proteins. A panel of multiple protein markers can be used to determine the disease status more accurately and informatively than using a single marker. Moreover, target analytes, such as disease markers, are often at very low concentrations in complex biofluids like blood. Many disease-related proteins exist at the low pg/mL concentration range in plasma while common proteins like albumin exist in 32 to 53 mg/ml, 9-10 orders of magnitude higher than those of the disease markers, making the detection and quantification of the disease markers like finding a needle in a haystack. A common method to meet the sensitivity and specificity requirements of multiplex biosensors is using antibody-based immunoassays. However, multiplexing with immunoassays presents challenges such as cross-reactivity and scaling limitations [1].

Antibody-microarray-based bioassays provide a platform for performing hundreds to thousands of immunoassays simultaneously on a single chip. Typically, a microarray spotter is used to spot antibodies (Abs) onto a substrate such as a glass slide for the fabrication of an antibody microarray. The size of each spot can range from tens to hundreds of micrometers in diameter, with (sub-) nanoliter per spot in volume, and each spot can host a separate assay. Immunoassays can be performed by spotting different antibodies in different spots. Common challenges with conventional microarray technologies include the optimization of spotting buffer for different Abs, irreproducible spot morphology, and high cost of the microarray spotter, all limiting broader applications of the antibody microarray technology. Moreover, when printing bioreagents with a microarray printer, the pico to nano liter volumes printed for each spot can evaporate very quickly due to their large surface area to volume ratio. To prevent drying, hygroscopic additives such as glycerol are often added; however, these additives can negatively impact the bioreactivity of the biomolecules. The printing ink characteristics relating to bioreactivity, evaporation issue, and viscosity are optimized carefully. Even with hygroscopic additives, evaporation is still often occurring and can result in poor spot morphology like coffee rings of the spots and impact the assay reproducibility [2]. Indeed, just a small amount of evaporation can create a coffee ring effect where reagents are pushed to the edge creating an uneven, banded deposition of reagents on the surface. In addition, conventional multiplex sandwich immunoassays are based on using a mixture of different detection antibodies, which results in undesired mixing between different antibodies and antigens that are not cognate to each other, in turn leading to potential cross-reactions and false positive signals [1]. For example, an irrelevant detection antibody could mistakenly bind with an antigen, a capture antibody, or another detection antibody that it should not bind. The number of possible false positive scenarios increases exponentially with scaling the number of simultaneous immunoassays limiting the scalability of microarrays [1]. These potential cross-reaction scenarios can be mitigated via avoiding unnecessary mixing of different reagents.

3D microarrays are microarrays that use nanostructures, hydrogels or paper membranes for more sensitive assays [4]. The increased surface area provided by the 3D structures increases the biomolecule binding capacity thus increasing the signal output. Conventionally when creating 3D Ab microarrays on a membrane, microarray printers are used similarly to 2D Ab microarray fabrication to print Abs onto the surface and the Abs will be immobilized onto the membrane by physical adsorption. Although promising, the applications of these 3D microarrays are still limited due to the requirement of a complex spotting instrument and potential irreproducible spot morphology.

Accordingly, there is a need for multiplex biosensors with high sensitivity and specificity that can be prepared on a scale without loss of quality.

### SUMMARY

The present application discloses compartmentalized arrays and methods of using the arrays for conducting multiplexed bioanalysis. The present application also discloses the methods for preparing the arrays. The arrays can be used, for example, in the fabrication and application of microarray-based biosensors with higher sensitivity.

In one aspect of the present application, to overcome challenges present in the art and enable broader applications of, for example, highly sensitive antibody-microarray-based biosensors, in this work, a membrane-based compartmentalized linker array, for example a nitrocellulose Compartmentalized Linker Array (nCLA), was developed to allow for the fabrication and application of 3D antibody-microarray-based biosensors via simple pipetting. The array uses the 3D structure of a membrane (approximately 150 μm thick) to facilitate higher assay sensitivity. However, the inherent antibody-adsorption property of the membrane can make it challenging to realize the compartmentalized linker array (CLA) concept. Unlike glass slides where linker molecules can be deposited directly and the glass background without linkers does not bind antibodies, membrane themselves can form a continuous layer of linker due to their antibody adsorption ability.

Accordingly, a novel “selective background de-activation” method was developed that can be used to achieve CLA concept on adherent substrates such as membranes, for example nitrocellulose membranes. The method enables higher signal-to-noise ratio and in turn higher assay sensitivity due to the high biomolecule binding capacity of the membrane. Specifically, the three cancer-related proteins tested all achieved a limit of detection under 2 μg/mL, hundreds of folds more sensitive than ELISA and twice more sensitive than the on-glass CLA. Compared to conventional microarrays fabricated via printing antibodies, the methods of the present application enable homogeneous micro-spot morphology, with intra-day immunoassay coefficient of variation (CV) of 2% and inter-day CV of 4%, both improved from the 13% CV with glass-based CLA, therefore improving assay reproducibility and reliability.

In another aspect of the present application, a reverse compartmentalized linker array (rCLA) was developed, which is a prepatterned microchip designed for creating bio-microarrays by pipetting the bioreagent solution (e.g., antibody, protein, cell) into microliter sized compartments where the bioreagent will self-assemble into the microarray pattern. This self-assembly is facilitated by functionalizing the chip's surface with linker molecules (e.g., aminosilane) and deactivating the background of the microarray while leaving microspots of active chemical linker to adhere pipetted bioreagents. This is done by first deactivating/blocking the entire surface with a blocker protein, such as BSA, followed by microarray printing a chemical to disrupt the aminosilane-bioreagent interaction and remove the BSA blocker molecules from the printed areas. This reverts the microarray spots to un-blocked and reactivate the aminosilane linkers within the micro-spots, which capable of binding bioreagent again.

The present application therefore includes: a method of preparing a compartmentalized membrane array comprising:
a) adding one or more barriers on a surface of a membrane to form one or more compartments;
b) printing a hydrophilic polymer solution in the one or more compartments on the surface of the membrane in a plurality of distinct regions;
c) adding a solution comprising a blocking reagent to the one or more compartments, wherein the blocking reagent binds to the membrane surface in regions other than the plurality of distinct regions; and
d) washing the membrane to remove the hydrophilic polymer solution and excess blocking reagent, if present, to form the compartmentalized membrane array.

In some embodiments, the membrane is a cellulose membrane, optionally a nitrocellulose membrane.

In some embodiments, the one or more barriers are hydrophobic barriers.

In some embodiments, the hydrophilic polymer solution is an aqueous polyethylene glycol (PEG) solution.

In some embodiments, the blocking reagent is bovine serum albumin (BSA).

In some embodiments, the hydrophilic polymer solution prevents or substantially reduces the blocking reagent from binding to the membrane in the plurality of distinct regions.

The present application includes a compartmentalized membrane array prepared using the above method and a package comprising a compartmentalized membrane array and a desiccant.

The present application also includes a method of preparing a reverse compartmentalized array comprising
a) coating a surface of a substrate with linker molecules to form a linker monolayer;
b) coating the linker monolayer with a blocking reagent solution to form a blocking reagent layer on the linker monolayer;
c) printing a solution of a blocking reagent remover on a plurality of distinct regions on the blocker reagent layer; and
d) washing the substrate to remove the blocking reagent remover and the blocking reagent on the plurality of distinct regions to form the reverse compartmentalized array,
   wherein the substrate comprises one or more barriers on the surface that form one or more compartments.

In some embodiments, the substrate is glass or nitrocellulose membrane.

In some embodiments, the blocking reagent is bovine serum albumin (BSA).

In some embodiments, the blocking agent remover is sodium dodecyl sulfate

(SDS).

In some embodiments, the one or more barriers are hydrophobic barriers.

The present application also includes a reverse compartmentalized array comprising:
a) a substrate comprising a surface coated with a linker molecule monolayer;
b) a blocking reagent layer on the linker molecule monolayer; and
c) a plurality of distinct regions in the blocker reagent layer wherein the blocking reagent layer has been removed to expose the linker molecule monolayer;
   wherein the substrate comprises one or more barriers on the surface that form one or more compartments.

The present application includes a use a compartmentalized membrane array or a reverse compartmentalized array of the present application for assaying a sample.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 shows a schematic illustration of an exemplary method of preparing and using the 3D membrane comprising a compartmentalized array of the present application (nCLA on FIG. 1) (scale not to size). Part 1: selective background de-activation process for nCLA preparation according to exemplary embodiments of the application. Part 2: preparation of exemplary antibody microarrays with the nCLA and multiplex immunoassay procedure.

FIG. 2 shows the results of a microarray study with an exemplary membrane comprising a compartmentalized array with fluorescently labeled antibody FITC-rabbit IgGs: (a) shows the spot intensity with the hydrophilic polymer solution of PEG 2% and 5%; and 50% glycerol according to exemplary embodiments of the application; (b) shows the results of the spot intensity as a result of a co-incubation duration of the hydrophilic polymer solution of 2% PEG with the blocking solution of 10% BSA according to exemplary embodiments of the application. Error bars of panel (a) indicate the standard error of the mean.

FIG. 3 shows the surface of the BSA-blocked areas on exemplary nCLA membrane: a) shows a contact angle of 110° for aqueous solutions after retrieving the BSA-blocked nCLA membrane from long-term storage; b) shows a contact angle of 40° for aqueous solutions after retrieving the BSA-blocked nCLA membrane from long-term storage and pre-wetting the membrane with PBS before the addition of the antibody solution; and c) shows intra-day and inter-day coefficient of variation (%) for exemplary nCLA membrane compared to aminosilane coated glass CLA (agCLA).

FIG. 4 shows (a) a comparison of the antibody microarray intensity prepared from membrane comprising an exemplary compartmentalized array of linker molecules of the present application (nCLA-middle columns), conventional antibody microarray printed on nitrocellulose (nitrocellulose-left columns), and agCLA with aminosilane linker microarray on glass (right columns), the left column in each one of the groups represents spot intensity and right column represents the background; (b) selective background de-activation results obtained by incubating an exemplary 2% PEG microarray with 10% FITC-labelled BSA showing the selective blocking of the background with minimal BSA binding to the spots. Error bars of panel (a) present on each bar indicate the standard error of the mean.

FIG. 5 shows the coffee ring effect of the printed droplet in the prior art printed microarrays.

FIG. 6 shows the intensities of the exemplary FITC-rabbit IgG microarray spots (a) and the background (b) after storage of the membrane at 4, −21 and 20° C. for 3 and 7 days.

FIG. 7 shows results of a storage test for arrays produced using an exemplary membrane comprising a compartmentalized array of linker molecules of the present application after storage at −20° C. via evaluating the antibody binding capacity of nitrocellulose microspots and background compared and normalized to freshly prepared slides over 240 days. Error bars indicate the standard error of the mean. FIG. 8 shows nCLA assay plots depicting fluorescence intensity of exemplary membranes of the application for (a) TNF-α, (b) EGFR, and (c) GM-CSF spiked in PBS, with concentrations ranging from 1 pg/mL to 100 ng/ml, diluted by a factor of 10, including a blank sample at 0 pg/mL. (d) Calculated limits of detection for each marker with nCLA. Error bars indicate the standard error of the mean. e) images of the multiplex assays.

FIG. 9 shows nCLA assay plots depicting fluorescence intensity of exemplary membranes of the application for (a) TNF-α, (b) EGFR, and (c) GM-CSF in 10% serum diluted in PBS, with concentrations ranging from 1 pg/mL to 100 ng/ml, diluted by a factor of 10, including a blank sample at 0 pg/mL. (d) Calculated limits of detection for each marker with nCLA. Error bars indicate the standard error of the mean.

FIG. 10 shows a schematic of an exemplary method for producing a reverse compartmentalized linker (rCLA on FIG. 10) with antibody added thereto.

FIG. 11 shows the stripping efficiency of exemplary chemicals for removing the blocking agent from the linkers in the preparation of rCLAs at pH 7 and pH 10.5 as measured through the reduction in fluorescence activity of FITC-BSA bound to aminosilane compared to positive control.

FIG. 12 shows the optimization of aminosilane linker reactivation using sodium dodecyl sulfate (SDS) at various concentrations as measured using the fluorescence intensity after attaching FITC-tagged antibodies and compared with a positive control of standard aminosilane-based antibody binding. Error bars represent standard error.

FIG. 13 shows fluorescence microarray scans of an exemplary rCLA made by microarray printing varying concentrations of SDS: a) 1.35% SDS, b) 0.45% SDS, c) 0.15% SDS, d) 0.05% SDS.

FIG. 14 shows fluorescence microarray scans comparing various exemplary techniques for washing away the microarray printed SDS: a) pipetted 22° C. wash buffer b) pipetted 10° C. wash buffer c) inserted rCLA chip parallel to the surface of 20 ml of wash buffer d) inserted rCLA chip perpendicular to the surface of 20 ml of wash buffer, e) flushed with squirt bottle at ~5 mL/s with wash buffer, f) flushed at 100 mL/s with DI water. Scale bar: 1 mm.

### DESCRIPTION OF VARIOUS EMBODIMENTS

### I. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

As used in this application, the singular forms “a”, “an” and “the” include plural references unless the content clearly dictates otherwise.

The term “distinct regions” as used herein refers to areas on a surface of the substrate, separated from each other and defined by a boundary having a predetermined shape and size.

The term “linker(s)” as used herein refers to any entity that is used to provide an attachment of a desired molecule to a substrate. The attachment can be via a chemical bond or via attractive forces. The linker can be part of the substrate itself or can be a chemical entity that is added to the substrate.

The term “array” as used herein refers to an arrangement of linker molecules or molecules of interest on a substrate surface.

The term “substrate” refers to any substance with a surface for forming an array.

The term “multiplexed assay” as used herein refers to an assay simultaneously measuring multiple analytes in a single experiment.

### II. Methods of the Application

### (i) Compartmentalized Membrane Arrays

The present application includes a method of preparing a compartmentalized membrane array comprising:
(a) adding one or more barriers on a surface of a membrane to form one or more compartments;
(b) printing a hydrophilic polymer solution in the one or more compartments on the surface of the membrane in a plurality of distinct regions;
(c) adding a solution comprising a blocking reagent to the one or more compartments, wherein the blocking reagent binds to the membrane surface in regions other than the plurality of distinct regions; and
(d) washing the membrane to remove the hydrophilic polymer solution and excess blocking reagent, if present, to form the compartmentalized membrane array.

In some embodiments, the membrane is cleaned before preparation of the array. In some embodiment, the membrane is washed with a stream of air to dry the membrane and remove any dust from the surface.

The membrane can be any 3D membrane that has high affinity to biomolecules. In some embodiments, the membrane is a nanostructure, hydrogel or a paper membrane. In some embodiments, the membrane is a paper membrane. In some embodiments, the membrane is a cellulose membrane. In some embodiments, the membrane is a nitrocellulose membrane. In some embodiments, the membrane is unsupported or supported on any suitable material, such as silicon, glass, plastics and the like. In some embodiments, the membrane is supported on glass.

The membrane can take a variety of configurations. This includes various sizes, shapes, pore sizes and thicknesses of the membrane. In some embodiments, the thickness of the membrane is between about 100 μm to about 250 μm, or about 150 μm. In some embodiments, the pore size is about 0.1 μm to about 1 μm, or about 0.45 μm. The size, the shape, the pore size and the thickness of the membrane is within the consideration of the person skilled in the art and the particular use of the array.

The one or more barriers are any suitable barriers that substantially eliminate or prevent mixing of different samples on the surface of the 3D membrane. In some embodiments, the one or more barriers are hydrophobic barriers. In some embodiments, the hydrophobic barriers are formed on the surface of the membrane with a hydrophobic barrier pen. In some embodiments, the hydrophobic barrier pen is peroxidase-anti peroxidase (PAP) pen.

In some embodiments, the one or more barriers are physical barriers. In some embodiments, the physical barriers are structures, separate or interconnected therebetween forming walls between the distinct regions on the surface of the membrane.

In some embodiments, the physical barriers are removable. In some embodiments, the physical barriers are non-removable. The non-removable physical barriers refer to physical barriers, fixed to the array and can be assembled by any method known in the art.

The size of the one or more compartments on the array can be the same or different and is within the consideration of the skilled person. An exemplary size of the compartments is 3 mm×3 mm. However, any other size of the compartments can also be used.

In some embodiments, the hydrophilic polymer solution comprises one or more hydrophilic and uncharged polymers, such as for example polyethylene glycol (PEG), glycerol and the like. In some embodiments, the hydrophilic polymer has weak interaction with the membrane and proteins.

In some embodiments, the hydrophilic polymer solution is an aqueous polyethylene glycol (PEG) or a glycerol solution. Any suitable Mw of PEG can be used, including, for example, but not limited to, PEG with a Mw between about 100 Da and about 100,000 Da. An exemplary Mw of the PEG is about 35,000 Da.

In some embodiments, the hydrophilic polymer solution is an aqueous polyethylene glycol (PEG) solution. In some embodiments, the PEG in the hydrophilic polymer solution is present in a concentration of about 1 w/v % to about 12 w/v %. In some embodiments, the PEG in the hydrophilic polymer solution is present in a concentration of about 1 w/v %, about 2 w/v %, about 5 w/v %, or about 10 w/v % and values therebetween. In some embodiments, the PEG in the hydrophilic polymer solution is present in a concentration of about 2 w/v %.

In some embodiments, the hydrophilic polymer solution is an aqueous glycerol solution. In some embodiments, concentration of glycerol in the hydrophilic polymer solution is about 40 w/v % to about 75 w/v %. In some embodiments, concentration of glycerol in the hydrophilic polymer solution is about 45 w/v %, about 50 w/v %, about 60 w/v %, or about 70 w/v % and values therebetween.

In some embodiments, the hydrophilic polymer solution is printed on the surface of the membrane using contact-based printing or noncontact-based printing. In some embodiments, the hydrophilic polymer solution is printed on the surface of the membrane using contact-based printing. In some embodiments, the contact-based printing is selected from micro-contact printing and pin spotting.

The amount of the printed spot can vary and is within the consideration of the person skilled in the art. An exemplary amount of the spot is about 1 nL. However, other amounts can also be used.

In some embodiments, the hydrophilic polymer solution printed on the membrane in the plurality of distinct regions temporarily blocks the membrane in said plurality of distinct regions.

In some embodiments, the membrane comprises at least two compartments and the plurality of distinct regions in each of the at least two compartments optionally comprise different arrangements of the distinct regions. In some embodiments, each distinct region of the plurality of distinct regions optionally has a different size. In some embodiments, one or more of the plurality of distinct regions within the compartments optionally have a different size. It is to be understood that the plurality of distinct regions within one or more compartments can have the same size which is different from the size of the plurality of distinct regions within another compartment.

In some embodiments, the plurality of distinct regions in each of the at least two compartments are optionally organized into different patterns or arrangements. In some embodiments, at least one of the different arrangements of the distinct regions comprises a gradient spacing between the distinct regions. It is to be understood that the gradient spacing can be between, rows, columns or both. In some embodiments, the gradient spacing allows for deposition of a gradient of bioreagent within a sub-array.

The size and the shape of the distinct regions can vary according to particular use of the application and is within the consideration of the person skilled in the art. In some embodiments, the plurality of distinct regions are spots. In some embodiments, the spots have a circular or a semi-circular shape.

In some embodiments, the plurality of distinct regions are spots having a diameter greater than 20 nm, greater than 40 nm, greater than 50 nm, greater than 80 nm or greater than 100 nm. In some embodiments, the plurality of distinct regions are spots having a diameter greater than 50 nm.

In some embodiments, the plurality of distinct regions are spots having a diameter greater than 500 nm, greater than 1 micron or greater than 1.5 micron. In some embodiments, the plurality of distinct regions are spots having a diameter greater than 1 micron.

In some embodiments, the blocking reagent solution comprises a blocking reagent and a buffer. In some embodiments, the blocking reagent is a protein blocking reagent. In some embodiments, the blocking reagent is bovine serum albumin (BSA) or succinic anhydride (SA). In some embodiments, the blocking reagent is BSA. In some embodiments, the buffer is phosphate-buffered saline (PBS) buffer.

In some embodiments, the blocking reagent is present in the blocking reagent solution in an amount of about 0.2% w/v to about 20% w/v. In some embodiments, the blocking reagent is present in an amount of about 0.5% w/v to about 15% w/v. In some embodiments, the blocking reagent is present in an amount of about 10% w/v.

In some embodiments, the blocking reagent solution is added to the one or more compartments with a pipette. In some embodiments, the blocking reagent solution is added to fully cover each compartment. Various amounts of the blocking reagent solution can be used based on the size of the compartments and are within the consideration of the skilled person. An exemplary amount of the blocking reagent solution in a compartment of 3 mm×3 mm is about 2 μL. However, other suitable amounts can also be used.

In some embodiments, the blocking reagent solution is allowed to incubate on the array for about 30 seconds to about 5 minutes. In some embodiments, the blocking reagent solution is allowed to incubate on the array for about 1 minute to about 2 minutes. In some embodiments, the blocking reagent solution is allowed to incubate on the array for about 1 minute, about 1.5 minutes, about 2 minutes, about 2.5 minutes or about 3 minutes.

In some embodiments, the blocking solution forms blocked background regions surrounding the plurality of distinct regions on the surface of the membrane.

In some embodiments, the hydrophilic polymer solution prevents or substantially reduces the blocking reagent from binding to the membrane in the plurality of distinct regions. In some embodiments, the hydrophilic polymer solution prevents or substantially reduces the blocking reagent from binding to the membrane in the plurality of distinct regions by limiting the amount of protein diffusing into the hydrophilic polymer solution spots due to weak interaction between the hydrophilic polymer present in the hydrophilic polymer solution and nitrocellulose. In some embodiments, the blocked background regions are deactivated surfaces on the membrane.

In some embodiments, the hydrophilic polymer solution is removed from the surface, thereby exposing the surface of the membrane in the plurality of distinct regions to form the compartmentalized membrane array. In some embodiments, the hydrophilic polymer solution is removed by washing the membrane with a suitable solvent, such as PBS. In some embodiments, the washing step further removes excess of blocking reagent. In some embodiments, the method further comprises drying the membrane array under a steam of air.

In the preparation method of the present application, compartmentalized membrane array is formed, in which the background of the membrane is selectively deactivated and reactive nitrocellulose regions are available to bind one or more desired reagents to the membrane.

The compartmentalized membrane array is selected from a microarray and a nanoarray. In some embodiments, the compartmentalized membrane array is a microarray. In some embodiments, the compartmentalized membrane array is a nanoarray.

In some embodiments, the compartmentalized membrane array is stored at a temperature below about 0° C. In some embodiments, the compartmentalized membrane array is stored in a temperature of about −20° C. In some embodiments, the compartmentalized membrane array is stored in a sealed bag, optionally with a desiccant. In some embodiments, the compartmentalized membrane array is stable for at least 8 months when stored a temperature of about −20° C.

In some embodiments, when the compartments on the membrane array are formed with removable barriers, said barriers can be removed after adding step (c) or after the washing step (d), and thus the compartmentalized membrane array can be stored without the barriers.

### (ii) Reverse Compartmentalized Array

The present application further includes a method of preparing a reverse compartmentalized array comprising:
(a) coating a surface of a substrate with linker molecules to form a linker monolayer;
(b) coating the linker monolayer with a blocking reagent solution to form a blocking reagent layer on the linker monolayer;
(c) printing a solution of a blocking reagent remover on a plurality of distinct regions on the blocker reagent layer; and
(d) washing the substrate to remove the blocking reagent remover and the blocking reagent on the plurality of distinct regions to form the reverse compartmentalized array,
   wherein the substrate comprises one or more barriers on the surface that form one or more compartments.

Substrates which can be used in the reverse compartmentalized array of the present application can be prepared from various materials and are within the consideration of the person skilled in the art. Examples of suitable substrates include, but are not limited to, silicon, glass, membranes such as a nanostructure, hydrogel or a paper membrane and rigid plastics, e.g. polytetrafluoroethylene, polypropylene, polystyrene, polycarbonate, and blends thereof, and mica. In some embodiments, the substrate is selected from glass, silicon, polystyrene, mica and cellulose membrane, such as nitrocellulose membrane. In some embodiments, the substrate is glass or nitrocellulose membrane.

The substrates can take a variety of configurations. This includes slide or plate configuration, such as a rectangular, square or disc configuration and may vary as required. The size and the shape of the substrate is within the consideration of the person skilled in the art and the particular use of the array.

The surface of the substrate can be smooth or substantially planar, or have periodic irregularities, i.e. a series of depressions forming wells, such as depressions or elevations, or have a porous surface, such as is found in porous glass or silica.

In some embodiments, the substrate is thoroughly washed and optionally activated prior to coating with the linker molecules using methods known in the art, such as acid washes and plasma treatment.

In some embodiments, the linker molecules are selected from an aminosilane, a poly-L-lysine, an epoxysilane, an aldehyde silane, a trichloro(alkyl) silane, and streptavidin. In some embodiments, the linker molecules are an aminosilane or an epoxy silane. In some embodiments, the aminosilane is ((3-aminopropyl)triethoxysilane). In some embodiments, the epoxysilane is 3-glycidoxypropyldimethoxymethylsilane (GPS).

In some embodiments, the substrate is coated with linker molecules by any known method, such as spraying, submersion in or printing of a printing solution comprising the linker molecules. In some embodiments, the printing solution comprises a printing buffer and one or more additives.

In some embodiments, the one or more compartments are configured to contain about 1 to about 10 microlitres of a solution. In some embodiments, the one or more compartments are configured to contain about 2 to about 3 microlitres of the solution.

In some embodiments, the blocking reagent solution comprises a blocking reagent and a buffer. In some embodiments, the blocking reagent is a protein blocking reagent. In some embodiments, the blocking reagent is bovine serum albumin (BSA) or succinic anhydride (SA). In some embodiments, the blocking reagent is BSA. In some embodiments, the buffer is phosphate-buffered saline (PBS) buffer.

In some embodiments, the blocking reagent is present in the blocking reagent solution in an amount of about 0.2% w/v to about 20% w/v. In some embodiments, the blocking reagent is present in an amount of about 0.5% w/v to about 15% w/v. In some embodiments, the blocking reagent is present in an amount of about 10% w/v.

In some embodiments, the blocking reagent solution is added to the printed array with a pipette. In some embodiments, the blocking reagent solution is added to fully cover each compartment. Various amounts of the blocking reagent solution can be used based on the size of the compartments and are within the consideration of the skilled person. An exemplary amount of the blocking reagent solution in a compartment of 3 mm×3 mm is about 2 μL. However, other suitable amounts can also be used.

In some embodiments, the blocking reagent solution is allowed to incubate for about 30 seconds to about 5 minutes to bind to the membrane surface in regions other than the plurality of distinct regions. In some embodiments, the blocking reagent solution is allowed to incubate for about 1 minute to about 2 minutes. In some embodiments, the blocking reagent solution is allowed to incubate for about 1 minute, about 1.5 minutes, about 2 minutes, about 2.5 minutes or about 3 minutes.

In some embodiments, the blocking agent forms blocking reagent layer on top of the linker monolayer substantially or entirely covering the monolayer.

In some embodiments, the solution of the blocking reagent remover is added to the blocking reagent layer to remove the blocking agent in a plurality of distinct regions on the surface of the substrate, unblocking the linker molecules on the substrate in the plurality of distinct regions. Thus, the linker molecules are un-blocked and reactivated in the plurality of distinct regions on the surface of the substrate and are thus capable of binding biomolecules in said regions.

In some embodiments, the solution of the blocking reagent remover is printed in a plurality of distinct regions on the surface of the substrate. In some embodiments, the solution of the blocking reagent remover is an aqueous printing solution.

In some embodiments, the solution of the blocking reagent remover has a pH of about 10 to about 11.

In some embodiments, the blocking agent remover is sodium dodecyl sulfate (SDS) or any other suitable detergent.

In some embodiments, the blocking agent remover is present in the solution of the blocking reagent remover in an amount of about 0.05% w/v to about 3% w/v. In some embodiments, the blocking agent remover is present in the solution of the blocking reagent remover in an amount of about 1.35% w/v.

In some embodiments, the solution of the blocking reagent remover is printed in a plurality of distinct regions on the blocking reagent layer using contact-based printing or noncontact-based printing. In some embodiments, the solution of the blocking reagent remover is printed on the blocking reagent layer using contact-based printing. In some embodiments, the contact-based printing is selected from micro-contact printing and pin spotting.

In some embodiments, the solution of the blocking reagent remover is printed on blocking reagent layer using noncontact-based printing. In some embodiments, the noncontact-based printing is inkjet printing.

In some embodiments, washing of the substrate to remove the blocking reagent remover and the blocking reagent on the plurality of distinct regions to form the reverser compartmentalized array is performed with water. In some embodiments, the washing is performed using a high flow rate of water, optionally using a squirt bottle.

The one or more barriers are any suitable barriers that substantially eliminate or prevent mixing of different samples on the surface of the substrate. In some embodiments, the one or more barriers are hydrophobic barriers. In some embodiments, the hydrophobic barriers are formed on the surface of the substrate with a hydrophobic barrier pen. In some embodiments, the hydrophobic barrier pen is peroxidase-anti peroxidase (PAP) pen.

In some embodiments, the one or more barriers are physical barriers. In some embodiments, the physical barriers are structures, separate or interconnected therebetween forming walls between the distinct regions on the surface of the substrate.

In some embodiments, the physical barriers are removable. In some embodiments, the physical barriers are non-removable. The non-removable physical barriers refer to physical barriers, fixed to the array and can be assembled by any method known in the art.

In some embodiments, the reverse compartmentalized array comprises at least two compartments and the plurality of distinct regions in each of the at least two compartments optionally comprise different arrangements of the distinct regions. In some embodiments, each distinct region of the plurality of distinct regions optionally has a different size. In some embodiments, one or more of the plurality of distinct regions within the compartments optionally have a different size. It is to be understood that the plurality of distinct regions within one or more compartments can have the same size which is different from the size of the plurality of distinct regions within another compartment.

In some embodiments, the plurality of distinct regions in each of the at least two compartments are optionally organized into different patterns or arrangements. In some embodiments, at least one of the different arrangements of the distinct regions comprises a gradient spacing between the distinct regions. It is to be understood that the gradient spacing can be between, rows, columns or both. In some embodiments, the gradient spacing allows for deposition of a gradient of bioreagent within a sub-array.

The size and the shape of the distinct regions can vary according to particular use of the application and is within the consideration of the person skilled in the art. In some embodiments, the plurality of distinct regions are spots. In some embodiments, the spots have a circular or a semi-circular shape.

In some embodiments, the plurality of distinct regions are spots having a diameter greater than 20 nm, greater than 40 nm, greater than 50 nm, greater than 80 nm or greater than 100 nm. In some embodiments, the plurality of distinct regions are spots having a diameter greater than 50 nm.

In some embodiments, the plurality of distinct regions are spots having a diameter greater than 500 nm, greater than 1 micron or greater than 1.5 micron. In some embodiments, the plurality of distinct regions are spots having a diameter greater than 1 micron.

The reverse compartmentalized array of the application is selected from microarray and nanoarray. In some embodiments, the reverse compartmentalized array is a microarray. In some embodiments, the reverse compartmentalized array is a nanoarray.

In some embodiments, the reverse compartmentalized array of the application is stored at a temperature below about 0° C. In some embodiments, the reverse compartmentalized array is stored in a temperature of about −20° C. In some embodiments, the reverse compartmentalized array is stored in a sealed bag, optionally with a desiccant. In some embodiments, the reverse compartmentalized array is stable for at least 8 months when stored a temperature of about −20° C.

In some embodiments, when the compartments on the reverse compartmentalized array are formed with removable barriers, said barriers can be removed after the washing step (d), and thus the compartmentalized membrane array can be stored without the barriers.

### III. Arrays of the Application

### (i) Compartmentalized Membrane Array

The present application includes a compartmentalized membrane array prepared using a method of the present application. In some embodiments, the compartmentalized membrane array comprises:
(a) one or more barriers on a surface of the membrane forming one or more compartments;
(b) a plurality of distinct regions on the surface of the membrane for linking of biomolecules, the distinct regions being located within the one or more compartments; and
blocking reagent on the surface of the membrane between and surrounding the plurality of distinct regions.

The membrane can be any 3D membrane that has high affinity to biomolecules. In some embodiments, the membrane is a nanostructure, hydrogel or a membrane. In some embodiments, the membrane is a paper membrane. In some embodiments, the membrane is a cellulose membrane. In some embodiments, the membrane is a nitrocellulose membrane. In some embodiments, the membrane is unsupported or supported by any suitable material, such as silicon, glass, plastics and the like. In some embodiments, the membrane is coated on paper or glass.

The membrane can take a variety of configurations. This includes various sizes, shapes, pore sizes and thicknesses of the membrane. In some embodiments, the thickness of the membrane is between about 100 μm to about 250 μm, or about 150 μm. In some embodiments, the pore size is about 0.1 μm to about 1 μm, or about 0.45 μm. The size, the shape, the pore size and the thickness of the membrane is within the consideration of the person skilled in the art and the particular use of the array.

The one or more barriers are any suitable barriers that substantially eliminate or prevent mixing of the different samples on the surface of the 3D membrane. In some embodiments, the one or more barriers are hydrophobic barriers. In some embodiments, the hydrophobic barriers are formed on the surface of the membrane with a hydrophobic barrier pen. In some embodiments, the hydrophobic barrier pen is peroxidase-anti peroxidase (PAP) pen.

In some embodiments, the one or more barriers are physical barriers. In some embodiments, the physical barriers are structures, separate or interconnected therebetween forming walls between the distinct regions on the surface of the membrane.

In some embodiments, the physical barriers are removable. In some embodiments, the physical barriers are non-removable. The non-removable physical barriers refer to physical barriers, fixed to the array and can be assembled by any method known in the art.

The size of the one or more compartments on the array can be the same or different and is within the consideration of the skilled person. An exemplary size of the compartments is 3 mm×3 mm. However, any other size of the compartments can also be used.

In some embodiments, the one or more compartments are configured to contain about 1 to about 10 microlitres of a solution. In some embodiments, the one or more compartments are configured to contain about 2 to about 3 microlitres of the solution.

In some embodiments, the blocking reagent is bovine serum albumin (BSA).

In some embodiments, the compartmentalized membrane array comprises at least two compartments and the plurality of distinct regions in each of the at least two compartments optionally comprise different arrangements of the distinct regions. In some embodiments, each distinct region of the plurality of distinct regions optionally has a different size. In some embodiments, one or more of the plurality of distinct regions within the compartments optionally have a different size. It is to be understood that the plurality of distinct regions within one or more compartments can have the same size which is different from the size of plurality of distinct regions within another compartment.

In some embodiments, the plurality of distinct regions in each of the at least two compartments are optionally organized into different patterns or arrangements. In some embodiments, at least one of the different arrangements of the distinct regions comprises a gradient spacing between the distinct regions. It is to be understood that the gradient spacing can be between, rows, columns or both. In some embodiments, the gradient spacing allows for deposition of a gradient of bioreagent within a sub-array.

The size and the shape of the distinct regions of the membrane can vary according to particular use of the application and is within the consideration of the person skilled in the art. In some embodiments, the plurality of distinct regions of the linker molecules are spots. In some embodiments, the spots have a circular or a semi-circular shape.

In some embodiments, the plurality of distinct regions are spots having a diameter greater than 20 nm, greater than 40 nm, greater than 50 nm, greater than 80 nm or greater than 100 nm. In some embodiments, the plurality of distinct regions are spots having a diameter greater than 50 nm.

In some embodiments, the plurality of distinct regions are spots having a diameter greater than 500 nm, greater than 1 micron or greater than 1.5 micron. In some embodiments, the plurality of distinct regions are spots having a diameter greater than 1 micron.

The compartmentalized membrane array is selected from a microarray and a nanoarray. In some embodiments, the compartmentalized membrane array is a microarray. In some embodiments, the compartmentalized membrane array is a nanoarray.

In some embodiments, the compartmentalized membrane array is comprised in a package, such as a sealed bag, optionally with a desiccant.

In some embodiments, when the compartments on the membrane array are formed with removable barriers, said barriers are removed after preparation and therefore the compartmentalized membrane array of the application does not have barriers.

### (ii) Reverse Compartmentalized Array

The present application includes a reverse compartmentalized array prepared using a method of the present application. In some embodiments, the reverse compartmentalized array comprises:
(a) a substrate comprising a surface coated with a linker molecule monolayer;
(b) a blocking reagent layer on the linker molecule monolayer; and
(c) a plurality of distinct regions in the blocker reagent layer wherein the blocking reagent layer has been removed to expose the linker molecule monolayer;
   wherein the substrate comprises one or more barriers on the surface that form one or more compartments.

Substrates which can be used in the reverse compartmentalized array of the present application can be prepared from various materials and are within the consideration of the person skilled in the art. Examples of suitable substrates include, but are not limited to, silicon, glass, membranes such as a nanostructure, hydrogel or a paper membrane and rigid plastics, e.g. polytetrafluoroethylene, polypropylene, polystyrene, polycarbonate, and blends thereof, and mica. In some embodiments, the substrate is selected from glass, silicon, polystyrene, mica and cellulose membrane, such as nitrocellulose membrane. In some embodiments, the substrate is glass or nitrocellulose membrane.

The substrates can take a variety of configurations. This includes slide or plate configuration, such as a rectangular, square or disc configuration and may vary as required.

The size and the shape of the substrate is within the consideration of the person skilled in the art and the particular use of the array.

The surface of the substrate can be smooth or substantially planar, or have periodic irregularities, i.e. a series of depressions forming wells, such as depressions or elevations, or have a porous surface, such as is found in porous glass or silica.

In some embodiments, the linker molecules are selected from an aminosilane, a poly-L-lysine, an epoxysilane, an aldehyde silane, a trichloro(alkyl) silane, and streptavidin. In some embodiments, the linker molecules are an aminosilane or an epoxy silane. In some embodiments, the aminosilane is ((3-aminopropyl)triethoxysilane). In some embodiments, the epoxysilane is 3-glycidoxypropyldimethoxymethylsilane (GPS).

In some embodiments, the one or more compartments are configured to contain about 1 to about 10 microlitres of a solution. In some embodiments, the one or more compartments are configured to contain about 2 to about 3 microlitres of the solution.

The one or more barriers are any suitable barriers that substantially eliminate or prevent mixing of different samples on the surface of the substrate. In some embodiments, the one or more barriers are hydrophobic barriers. In some embodiments, the hydrophobic barriers are formed on the surface of the substrate with a hydrophobic barrier pen. In some embodiments, the hydrophobic barrier pen is peroxidase-anti peroxidase (PAP) pen.

In some embodiments, the one or more barriers are physical barriers. In some embodiments, the physical barriers are structures, separate or interconnected therebetween forming walls between the distinct regions on the surface of the substrate.

In some embodiments, the physical barriers are removable. In some embodiments, the physical barriers are non-removable. The non-removable physical barriers refer to physical barriers, fixed to the array and can be assembled by any method known in the art.

In some embodiments, the reverse compartmentalized array comprises at least two compartments and the plurality of distinct regions in each of the at least two compartments optionally comprise different arrangements of the distinct regions. In some embodiments, each distinct region of the plurality of distinct regions optionally has a different size. In some embodiments, one or more of the plurality of distinct regions within the compartments optionally have a different size. It is to be understood that the plurality of distinct regions within one or more compartments can have the same size which is different from the size of the plurality of distinct regions within another compartment.

In some embodiments, the plurality of distinct regions in each of the at least two compartments are optionally organized into different patterns or arrangements. In some embodiments, at least one of the different arrangements of the distinct regions comprises a gradient spacing between the distinct regions. It is to be understood that the gradient spacing can be between, rows, columns or both. In some embodiments, the gradient spacing allows for deposition of a gradient of bioreagent within a sub-array.

The size and the shape of the distinct regions can vary according to particular use of the application and is within the consideration of the person skilled in the art. In some embodiments, the plurality of distinct regions are spots. In some embodiments, the spots have a circular or a semi-circular shape.

In some embodiments, the plurality of distinct regions are spots having a diameter greater than 20 nm, greater than 40 nm, greater than 50 nm, greater than 80 nm or greater than 100 nm. In some embodiments, the plurality of distinct regions are spots having a diameter greater than 50 nm.

In some embodiments, the plurality of distinct regions are spots having a diameter greater than 500 nm, greater than 1 micron or greater than 1.5 micron. In some embodiments, the plurality of distinct regions are spots having a diameter greater than 1 micron.

The reverse compartmentalized array of the application is selected from microarray and nanoarray. In some embodiments, the reverse compartmentalized array is a microarray. In some embodiments, the reverse compartmentalized array is a nanoarray.

In some embodiments, the reverse compartmentalized array is comprised in a package, such as a sealed bag, optionally with a desiccant

In some embodiments, when the compartments on the reverse compartmentalized array are formed with removable barriers, said barriers are removed after preparation and therefore the reverse compartmentalized array of the application does not have barriers.

### IV. Methods of Using the Arrays of the Application

The present application includes a use of the arrays of the present application for assaying a sample.

Therefore, present application also includes a method of assaying a sample comprising:
adding a bioreagent solution to the plurality of distinct regions of a compartmentalized membrane array or a reverse compartmentalized array of the present application under conditions for the one or more reagents to bind to the membrane or linker molecules, respectively, in the plurality of distinct regions;
adding a sample solution containing an analyte to the plurality of distinct regions; and
reading the assay to assay the sample.

In some embodiments, the membrane of the compartmentalized membrane array is pre-wetted before the addition of the bioreagent solution. In some embodiments, the membrane is pre-wetted with a buffer, such as a PBS buffer.

The bioreagent that can be used in the methods of the present application is any bioreagent known in the art, or mixtures thereof. In some embodiments, the bioreagent is selected from antibodies, proteins, nucleic acids, cells, extracellular vesicles (EVs) and exosomes, or a mixture thereof.

In some embodiments, the bioreagent(s) are bound onto the membrane or the linker molecules by covalent bonds. In some embodiments, the bioreagent(s) are bound onto the membrane or the linker molecules by non-covalent bonds, e.g attractive forces. In some embodiments, the bioreagent(s) are added for incubation with the membrane or linker molecules.

In some embodiments, the bioreagent solution comprises a buffer. In some embodiments, the buffer is a phosphate-buffered saline (PBS). Concentration of the bioreagent in the bioreagent solution can vary based on, for example, the type of the bioreagent, the buffer solution and the intended use and is within the consideration of the person skilled in the art. Exemplary concentrations of the bioreagent in the buffer solution is between about 5 μg/mL to about 100 μg/mL.

In some embodiments, the incubation of the bioreagent(s) is under a humidified environment, optionally for about 1 hour. In some embodiments, the incubation of the bioreagent(s) is for about 20 min to about 1.5 h. In some embodiments, an optional washing step is performed after each incubation, for example using a suitable solvent, such as PBS. In some embodiments, the solutions and the reagents are added to the arrays using a pipette.

In some embodiments, the sample solution containing the analyte is selected from cell media, blood, urine, saliva, mucus, and other complex biofluids. The amount of the sample solution added to the array can vary depending, for example, on the size of the compartment, the type of the sample and the intended use and is within the consideration of the person skilled in the art. Exemplary amount of the sample solution is between about 1 μL to about 5 μL.

Analytes that can be detected in the assay according to the method of the present application are any analytes known in the art. Examples of suitable analytes include but are not limited to antigens, antibodies, nucleic acids, proteins, small molecules, hormones, receptors, ligands, extracellular vesicles (EVs), exosomes and the like. In some embodiments, the analyte is selected from antibodies, proteins, nucleic acids, cells, extracellular vesicles (EVs), exosomes, and combinations thereof. In some embodiments, the analyte is the antibody.

The assay that can be conducted using the array and methods of the present application are any bioassays known in the art. Examples of assay include but are not limited to immunoassay, proteomic assay and the like. In some embodiments, the assay is immunoassay. It is understood that any assay conducted for diagnostic, drug screening or research can be conducted using the array and methods of the present application.

In some embodiments, the assay is a multiplexed assay. In some embodiments, the multiplexed assay is performed for simultaneous detection of multiple analytes. In some embodiments, the multiplexed assay is multiplexed immunoassay. In some embodiments, the multiplexed assay is multiplexed sandwich immunoassay.

In some embodiments, the assay can be read using any suitable detection apparatus such as fluorometer, spectrometer, camera and the like. In some embodiments, the detection method utilized by the detection apparatus is selected from laser induced luminescence, FRET (fluorescence resonance energy transfer), fluorescence polarization, transmittance, fluorescence anisotropy, raman spectroscopy or color change.

In some embodiments, a 3D microarray comprising the compartmentalized membrane array of the application provides higher signal intensity compared to a 2D microarray. In some embodiments, the signal to background ratio of the 3D microarray is 2 times higher compared to 2D microarray.

In some embodiments, the 3D microarray comprising the compartmentalized membrane array of the application provides homogeneous spot morphology without coffee ring effect. The homogeneous spot morphology enabled by the 3D microarray comprising the compartmentalized membrane array of the application contributes to more reproducible microarray-based bioassays.

The present application also includes a use of an array of the present application for imaging. In some embodiments, imaging includes using the array of the present application with microscopes, environment-control chamber for cell analysis, surface plasmon resonance spectrometers.

The following non-limiting examples are illustrative of the present application.

### EXAMPLES

### Example 1

### Material and Methods

### Materials

Glycerol, phosphate-buffered saline (PBS) tablets, bovine serum albumin (BSA), Tween 20 viscous liquid, polyethylene, Whatman nitrocellulose membrane (0.45 μm pore size), poly-ethylene glycol (PEG) at the Mw of f 35,000 Da, (3-aminopropyl)triethoxysilane (APTES), polystyrene Petri dishes, fluorescein isothiocyanate (FITC)-conjugated BSA, and peroxidase-anti peroxidase (PAP) pen were purchased from Sigma Aldrich. Polyclonal goat anti-human epidermal growth factor receptor (EGFR) IgG cAb, monoclonal mouse anti-human granulocyte-macrophage colony-stimulating factor (GM-CSF) IgG capture antibody (cAb), monoclonal mouse anti-human tumour necrosis factor-alpha (TNF-α) IgG R cAb, goat anti-human EGFR IgG biotinylated dAb, mouse anti-human GM-CSF IgG biotinylated detection antibody (dAb), mouse anti-human TNF-α IgG biotinylated dAb, recombinant human GM-CSF, recombinant human EGFR, and recombinant human TNF-α were purchased from R&D Systems. Pooled human plasma, FITC-conjugated streptavidin, goat anti-rabbit immunoglobulin G (IgG), and FITC-labeled rabbit anti-goat IgG were purchased from Cedarlane.

### Fabrication of Compartmentalized Linker Array

To make the compartments on the nitrocellulose membrane, 4 mm×4 mm compartments (inside area 3 mm×3 mm) were made with a PAP pen on a nitrocellulose membrane mounted on a polystyrene petri dish to create hydrophobic barriers and eliminate reagent mixing. The membrane was then brought briefly under a stream of air to dry and remove any dust from the surface. A pin microarray spotter μArrayer (purchased from LabNEXT) was used to print a solution of 2% PEG dissolved in deionized water. After printing, 2 μL of 10% BSA in PBS was added to each compartment and incubated for 1 minute to selectively block the nitrocellulose background (non-PEG region). The membrane was then washed three times with PBS, 3 minutes on the first two washes and 24 minutes on the third wash to remove PEG and excess BSA, followed by drying under a stream of air.

An aminosilane on glass compartmentalized linker array (agCLA) was created following the procedure demonstrated before [3]. An aminosilane solution containing 1% APTES, 20% glycerol, and water was printed onto a cleaned glass slide compartmentalized with a 3 mm×3 mm pap pen grid. Shortly before removal, 10% BSA was pipetted into the compartments followed by washing with PBS containing 0.05% Tween-20 (PBST), PBS and rinsing with deionized (DI) water then drying under a stream of air.

Storage of nCLA

To store the nCLA, the membranes were placed in sealed plastic bags with desiccant. The packaged nCLAs were then placed into a freezer at −20 oC.

To prepare the stored nCLA for an immunoassay, the membranes were retrieved from the freezer and left at room temperature for 15 minutes before the package was opened to reacclimate the membrane and avoid condensation formation.

### Formation of Ab Microarrays and Performing Multiplexed Sandwich Immunoassays

Ab microarrays were fabricated by pipetting 2 μL of solutions of Abs in PBS into each compartment, 10 μg/mL for FITC-rabbit IgGs and 40 μg/mL for the sandwich immunoassay. The membrane was then incubated in a humidified petri dish for 1 hour. The membrane was washed two times with PBST and one time with PBS, for 3 min each time.

After forming the Ab microarrays, the following steps were used to conduct the multiplexed sandwich immunoassay for simultaneously measuring EGFR, TNF-α, and GM-CSF on the nCLA. 2 μL of 1% BSA was pipetted into each compartment and incubated for 30 minutes for another round of blocking. Then the membrane was washed once in PBS for 3 minutes. 2 μL of the samples of either diluted serum or PBS with spiked antigen concentrations of 0 pg/mL, 1 pg/mL, 10 pg/mL, 100 pg/mL, 1 ng/ml, 10 ng/ml, and 100 ng/ml were added to each compartment and incubated for 1 hour. The membrane was then washed two times with PBST, and once in PBS for three minutes, followed by incubating the compartments with solutions containing 5 μg/mL of each assay's associated biotinylated detection antibodies for 1 h. It was then washed two times with PBST and once with PBS, 3 min each time, and incubated with a FITC-streptavidin solution at 2 μg/mL for 20 min. After that, the membrane was washed two times with PBST, 3 min each time, once with PBS, and once briefly with DI water, followed by drying under a stream of air.

### Signal Acquisition and Analysis

A fluorescence microscope (Nikon Eclipse Ti) with a blue excitation filter at a wavelength of 420 nm to 495 nm was used to image the nCLA membrane. Image J software was used for extracting fluorescent signal intensities. The limit of detection (LOD) for each antigen was calculated using MATLAB software to obtain the interpolation of the value calculated by adding the standard deviation of the 0 pg/mL antigen concentration and its mean value from replicates as in Eq. 1 below. This value is then used in a semi-logarithmic interpolation shown in Eq. 2 below. MATLAB software was also used to make graphs. Error bars on the graphs represent the standard error. The lines of best fit for the standard curves were created using a four-parameter logistic regression [5].

$${y}_{LOD} = 3 * {σ}_{x = 0} + {μ}_{x = 0} \qquad Eq . 1$$

$${x}_{LOD} = exp ( ln ( {x}_{1} ) + ( {y}_{LOD} - {y}_{1} ) * \frac{ln ( {x}_{1} ) - ln ( {x}_{2} )}{{y}_{1} - {y}_{2}} ) \qquad Eq . 2$$

### Results and Discussion

nCLA Procedure

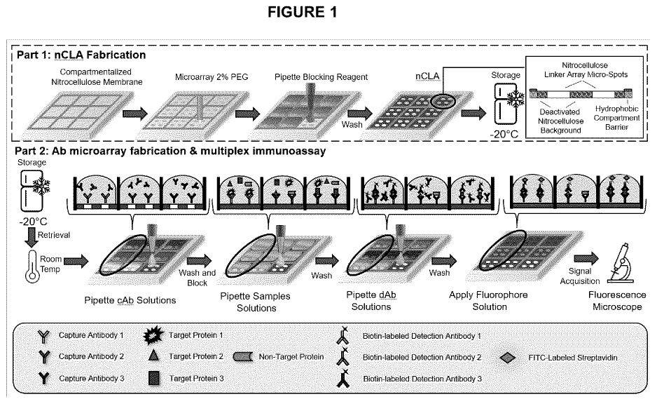

The fabrication, storage, and immunoassay process of an exemplary compartmentalized membrane array of the present application (nCLA) is illustrated in FIG. 1, identified as nCLA on the figure. The nCLA is a simple-to-implement platform for creating 3D microarrays. Conventional methods for creating 3D microarrays often involve either creating a 3D surface with lithography techniques or relying on a complex microarray printer to print onto 3D surfaces like cellulose or nitrocellulose membranes. Either way requires specialized instruments and expertise for the platform fabrication, unavailable for most biological and biomedical research laboratories. Additionally, microarray printing of biomolecules on nitrocellulose membranes often results in erratic and uneven spot morphology, decreased biomolecular activity, and requires optimization of the printing buffer for each different bioreagent. The compartmentalized membrane array of the present application tackles these challenges by fabricating antibody microarrays through pipetting microliters of antibodies in their conventional buffer solutions from the end-user's side, which provides uniform spot morphology and well-maintained bioactivity, without the need for any specialized instrument or expertise.

For the implementation of the nCLA concept, inherent reactivity of the nitrocellulose membrane was a challenge. Even without any additional surface functionalization, the nitrocellulose can bind antibodies by physical adsorption across the whole membrane. However, in the concept of nCLA, microarrays of reactive nitrocellulose spots are needed. Therefore, a novel approach, named “selective background de-activation” to create reactive nitrocellulose microarrays via de-activating the non-microarray background nitrocellulose was developed. The de-activation process involved using a concentrated blocking reagent, i.e., 10% BSA alongside a hydrophilic solution, i.e., PEG with a relatively low concentration at 2% (Part 1 of FIG. 1). First, the PEG ink was printed onto the compartmentalized nitrocellulose membrane using a pin microarray printer, approximately 1 nL per spot. The 2 μL BSA blocker was then applied to the nitrocellulose with a pipette to cover each compartment. During a short co-incubation of the PEG microarray and the BSA solution, the PEG prevented the BSA molecules from binding to nitrocellulose in the spotted area by limiting the amount of protein diffusing into the PEG spots via weak interaction of PEG with nitrocellulose, as well as steric hindrance to prevent BSA-nitrocellulose interaction. To achieve effective de-activation with this short co-incubation, 10% BSA was used, a higher concentration than commonly used BSA blocking solution in conventional immunoassays [6, 7]. After the short co-incubation, PEG and excessive BSA molecules were washed away, leaving the background nitrocellulose deactivated by the BSA blocker and the microarrayed spots as reactive nitrocellulose ready for binding biomolecules. The nCLA platform was then stored in a freezer with desiccant, through which the platform fabrication process can be separated from the immunoassay procedure on the end-users' side, allowing for technology dissemination.

To fabricate antibody microarrays with the nCLA for conducting multiplex immunoassays, the membrane was retrieved from the freezer and allowed to acclimate to the ambient temperature before the package was opened, to avoid condensation on the membrane (Part 2 of FIG. 1). Each compartment of the nCLA was then incubated with capture antibodies, samples, detection antibodies, and fluorophores by pipetting. Compartmentalization kept different reagents from undesired mixing by confining cognate antigens and antibodies to a specific compartment, thus reducing the chance of cross reactions between different antibodies and antigens. A sandwich assay format with the use of a capture and a detection antibody was chosen, in which the chance of the non-cognate component having affinity for both antibodies is low thus providing a high specificity comparable to single-plex sandwich assays. With the decreased concern of cross reactions, the nCLA was easily scaled for multiplex immunoassays.

### Optimization of the Selective Background De-Activation Procedure

When selecting the hydrophilic ink to microarray for the selective background de-activation process, 5 ink solutions were compared, including 50% and 70% glycerol in DI water as well as 2%, 5%, and 10% PEG in DI water. Glycerol and PEG possess both hydrophilic and uncharged molecular properties, allowing them to have low interaction with proteins and nitrocellulose. The 70% glycerol and 10% PEG solutions were immediately eliminated from the selection as their viscosities were too high for the microarray printer to dispense.

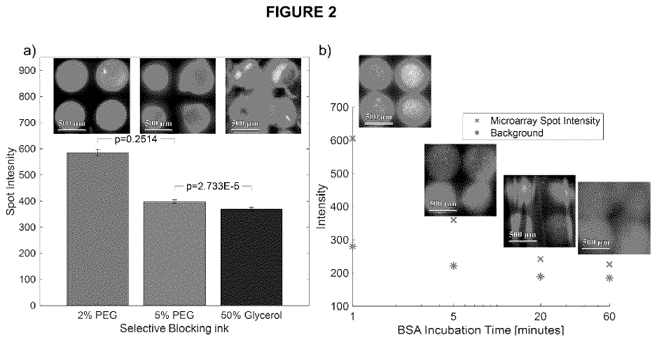

To optimize the selective background de-activation system and verify its ability to form antibody microarrays, fluorescently labeled antibody FITC-rabbit IgGs were used to form microarrays on the nCLAs made with different conditions for comparison. As shown in FIG. 2a, 2% PEG provided the most uniform spot morphology, cleaner background, and the greatest spot intensity from the FITC-rabbit IgG microarrays, therefore was selected to proceed.

Next, the co-incubation duration of the 10% BSA and the 2% PEG microarray was optimized. 1, 5, 20, and 60 minutes were tested, and verified the results by forming an antibody microarray with fluorescently labeled IgG. As shown in FIG. 2b, although the efficacy of the background blocking slightly increased with longer co-incubation, the spot intensities significantly decreased over time, probably because with longer co-incubation, more BSA molecules diffused into the PEG microarray spots and bind with the nitrocellulose substrate. Additionally, as time increased the quality of the spots' morphology also decreased and blurred into the background, which might be caused by the stronger interaction between PEG and nitrocellulose over time. Based on these results, the 1-minute co-incubation duration was selected.

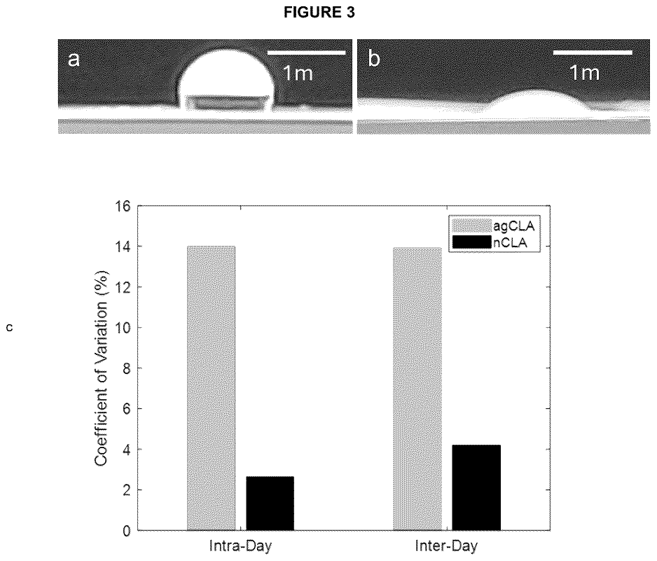

During the initial test of the repeatability of the nCLA, it was observed that the inter-day coefficient of variation (CV) was very high at 52%. During that time, it was noticed that the surface of the blocked areas became mildly hydrophobic, taking much longer time to absorb the pipetted solution than that of an untreated nitrocellulose membrane. The amount of solution absorbed throughout the compartment was not consistent. This hydrophobicity was particularly strong after retrieving the BSA-blocked nCLA membranes from long-term storage, with a contact angle of 110° for aqueous solutions (FIG. 3a). While not wishing to be limited by theory, a potential mechanism of this effect may be that in a non-aqueous environment, the hydrophilic and hydrophobic forces maintaining the BSA protein structure altered, resulting in exposure of hydrophobic aminoacids which increased the overall hydrophobicity of the surface [8]. Additionally, during evaporation, polymerization of the BSA blocking layer may create a water-insoluble matrix around the nitrocellulose fibers further decreasing its absorbent ability [9]. To mitigate this issue, the membrane was pre-wetted before the addition of the antibody solution with the goal of rehydrating the proteins to return to the proteins' original shape with mostly hydrophilic aminoacids exposed. 1 μL of PBS was pipetted into each compartment and incubated for 5 minutes to allow the wetness to evenly disperse throughout the compartment. Once pre-wetted, the contact angle was reduced to approximately 40° (FIG. 3b). The applied reagent was absorbed quickly and evenly into the compartment. This facilitated a lower inter-day CV of 4% alongside an intra-day CV of 2% (FIG. 3c). With the pre-wetting of the membrane, the nCLA provides an improved reproducibility compared to the aminosilane coated glass CLA (agCLA) which was shown to have an inter-day and intra-day CV of 13% [3].

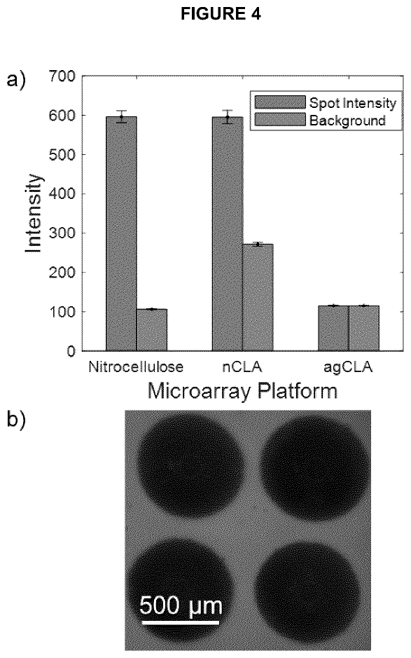

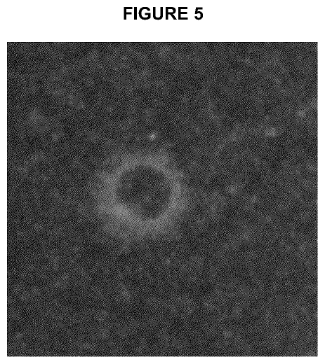

After optimization, the efficiency of the antibody microarray fabrication from the nCLA was confirmed by comparing it to a standard printed antibody microarray on a nitrocellulose membrane and an antibody microarray formed using agCLA imaging using the same exposure on the fluorescent microscope for equal comparison [3]. Compared with the agCLA, a 2D microarray, the 3D nCLA provided much higher antibody binding quantities as shown by the increased intensity from the bound FITC-labelled antibodies in FIG. 4a. The signal to background ratio of the nCLA was over 2 while the agCLA was only 1.03. Although the agCLA signal-to-background ratio improved with higher exposure time under the microscope, the nCLA's and nitrocellulose's high fluorescence quickly oversaturated the photodetector with longer exposure durations. The nCLA bound as many antibodies as a conventional printed microarray on nitrocellulose, demonstrating the effectiveness of the selective background-blocking process of the present application.

The selectivity of the BSA binding to the nitrocellulose background rather than the micro-spots is shown in FIG. 4b. The fluorescent FITC-labelled BSA bound to the background while the low fluorescence of the spots shows negligible BSA binding in the spots. The area spotted with 2% PEG provided a sharp edge between the spots and the de-activated background. The uniform micro-spots facilitated a homogenous antibody binding, as shown in the fluorescence image in FIG. 2a. An advantage of the microarrays, such as for example antibody microarrays, made with compartmentalized membrane array of the present application over those made by printing nanoliter antibodies is the homogeneous spot morphology without the coffee ring effect [2]. The coffee ring effect seen in many printed microarrays occurs as evaporation of the printed droplet pushes the reagents in the fluid to migrate to the edge of the droplet causing a ring or band of biomolecule binding rather than a continuous spot [2], seen in FIG. 5. The homogeneous spot morphology enabled by compartmentalized membrane array of the present application solves this long-standing challenge and can contribute to more reproducible microarray-based bioassays.

Storage of the nCLA

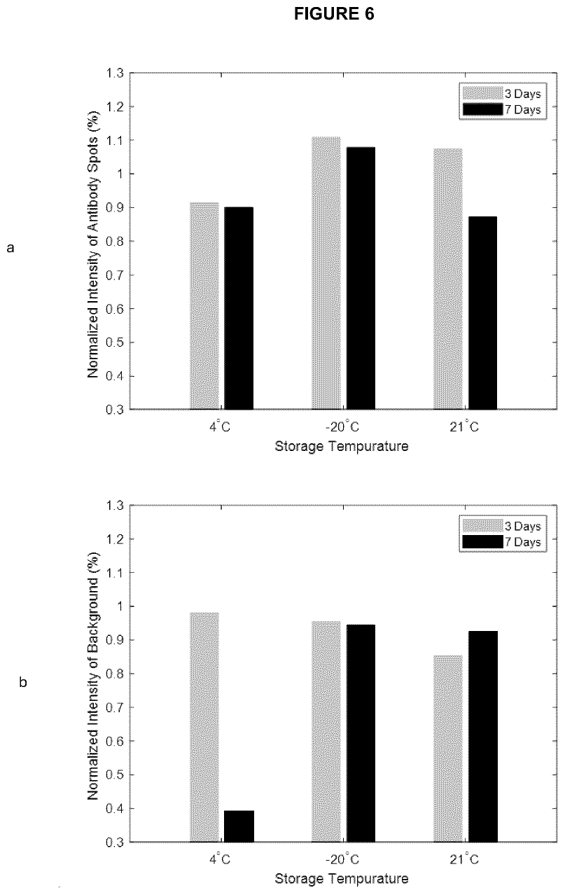

Storage of the nCLA is one of the parameters of the overall function as separation of the nCLA fabrication and the immunoassay process facilitates potential technology dissemination for fabricating 3D antibody microarrays without the need for a microarray printer. Commercially available nitrocellulose membranes are generally stable to store at room temperature in an unsealed container; however, the addition of a blocking reagent to the membrane changes how the storage environment affects the membrane's reactivity. As the BSA protein structure decays over time, residues may lead to a more reactive background region, affecting the performance of nCLA. To explore the storage conditions, first, short-term storage of 3 and 7 days of the nCLA was tested at −20° C., 4° C., and 21° C. in sealed plastic bags with desiccant. The stored membranes were then compared to an nCLA freshly made on the day of testing in terms of their ability to create an antibody microarray. The intensities of the antibody spots from the stored nCLA were normalized using those from the fresh nCLA made on the same day. FIG. 6 shows the intensities of the FITC-rabbit IgG microarray spots and the backgrounds after the storage test. The −20° C. provided the best antibody spot intensities compared to the other storage modalities (FIG. 6a). The BSA blocking was ineffective with the 4° C. after 7 Days, while the 21° C. and −20° C. had slightly diminished efficacy but were stable between the 3 and 7 days (FIG. 6b). With these results, the −20° C. storage condition was used for long-term storage tests.

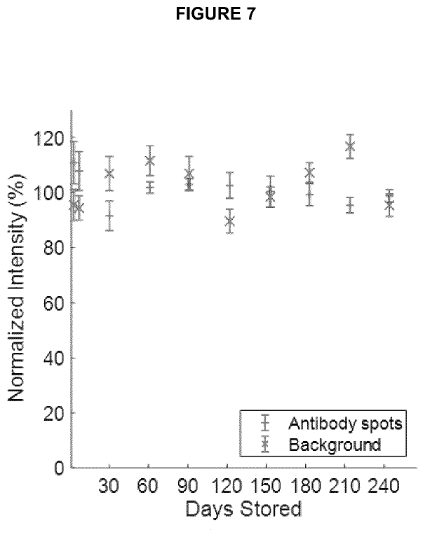

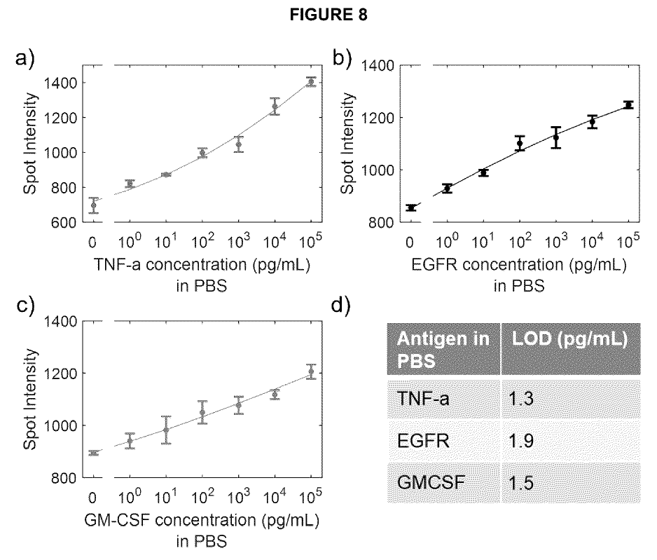

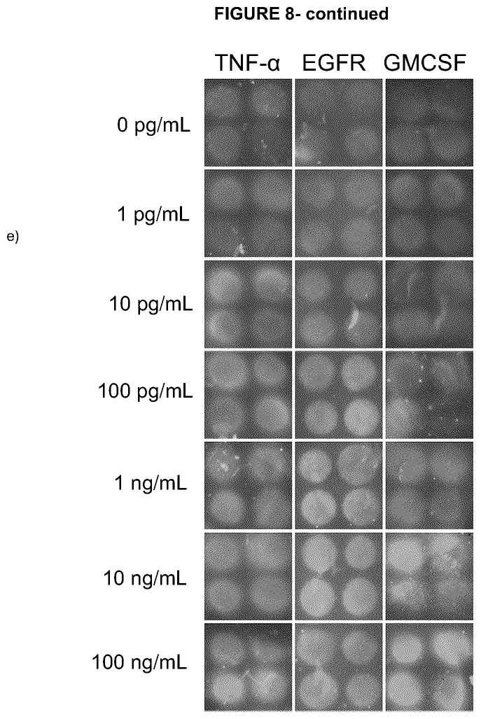

To test the long-term storage of the nCLA, 10 nCLA membranes were prepared and tested monthly. FIG. 7 shows the nCLA's long-term ability to create antibody microarrays after storage at −20° C. The antibody microarrays fabricated from the stored nCLAs and the blocked background intensity showed little variation from the nCLAs freshly prepared on the same day.

Multiplex Immunoassay Using nCLA

To demonstrate the application of the nCLA for sensitive multiplex bioassays, a multiplex sandwich immunoassay targeting three cancer-related protein markers: TNF-α, EGFR, and GM-CSF was conducted. The standard curves were made by measuring serial dilutions of proteins spiked in PBS. As the concentration of the protein marker increases, more target proteins bind to the capture antibody, leading to an increase in bound detection antibody and a higher number of fluorophores attached to the membrane, which ultimately results in a stronger fluorescent signal. The LODs of each marker were then compared to the LODs found with a conventional ELISA.

Conventionally, cross-reactivity plagues many multiplexing immunoassay platforms as different antibodies and antigens mix during the process leading to potential false positive results [1]. Antibodies may mistakenly bind to other molecules with similar structures to the epitope of the target antigen. In conventional antibody microarray-based multiplex sandwich immunoassays where detection antibodies are applied as a mixture, the chances of cross reactions grow exponentially with each additional target [1]. The potential cross reactions with the nCLA are mitigated, for example, through the use of microliter compartments where each compartment hosts cognate capture antibody, antigen, and detection antibody for an independent immunoassay without the risk of mixing different reagents.

In the multiplex immunoassay with the nCLA, known concentrations of recombinant proteins, TNF-α, EGFR, and GM-CSF, were spiked into PBS. EGFR is a transmembrane protein whose function relates to cell growth, division, and cell death resistance. Upregulation of EGFR can indicate diseases such as certain breast cancers, lung cancers, or glioblastomas [9]. Because of EGFR's presence and role in metastasis in many cancers, it is often the target of many cancer drugs. EGFR can be found in serum at an average concentration of 7771 pg/mL in healthy individuals [11]. TNF-α is a pro-inflammatory cytokine found soluble in blood and as a transmembrane protein that plays major roles in the development and pathology of many diseases such as some cancers, auto-immune inflammatory diseases and nephropathy. Increased levels may predict tumour cell growth and metastasis and predict prognosis. The normal expression level of TNF-α in serum is less than 8.1 pg/mL [12]. GM-CSF, a cytokine, is a part of pro-inflammatory pathways secreted by various immune cells. Upregulated GM-CSF is associated with colorectal cancer, head and neck squamous cell carcinoma, bladder, and brain and glioblastoma. The normal range of GM-CSF in serum is 20 to 100 pg/mL [13]. Sensitive, multiplex assays are essential for finding early changes in these disease markers for early diagnostics and prognosis.

The results of the multiplex assay are shown in FIG. 8 a-d with images of the microarrays shown in FIG. 8 e. In a conventional ELISA using the same reagents, the LODs of TNF-α, EGFR, and GM-CSF were 331.6 pg/mL, 58.3 pg/mL, and 14.6 pg/mL, respectively [4]. The agCLA had LODs of 3.5 pg/mL, 2.5 pg/mL, and 2.2 pg/mL for TNF-α, EGFR, and GM-CSF, respectively [3]. The nCLA was approximately hundreds of folds more sensitive than ELISA and twice more sensitive than the agCLA with LODs of 1.3 pg/mL, 1.9 pg/mL, and 1.5 pg/mL for TNF-α, EGFR, and GM-CSF respectively, benefited from the 3D structure of the nitrocellulose and more homogeneous spot morphology. With these LODs, the nCLA is able to detect the typical concentration of each protein in both healthy individuals and cancer patients.

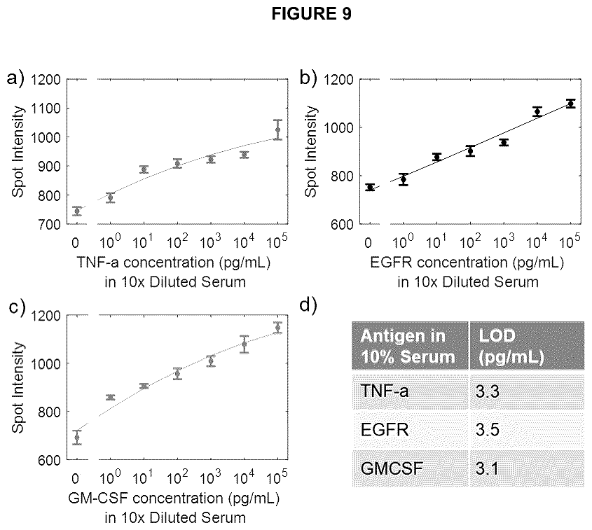

To mimic protein quantification with real complex biofluid samples, varying concentrations of the recombinant proteins EGFR, TNF-α, and GM-CSF in normal pooled human serum diluted with PBS were spiked (FIG. 9). When performing a multiplex assay on the nCLA with serum, residues from the serum were unable to be washed off from the membrane and created fluorescent artifacts on the membrane surface which interfered with the signal extraction from the microarray spots. To overcome this challenge, rather than imaging the top side of the membrane (the side to which the reagents were added), the images were taken from the backside of the membrane. The membrane appeared to act as a filter and the artifact-inducing residues in the serum did not penetrate to the backside of the membrane. This left the backside of the membrane with minimal artifacts from complex samples for signal acquisition. The LODs of TNF-α, EGFR, and GM-CSF were 3.5 pg/mL 3.3 pg/mL, and 3.1 pg/mL, respectively, demonstrating the feasibility of nCLA for highly sensitive multiplex protein quantification in complex biofluids.

### CONCLUSION

A novel platform has been developed to allow for the broader application of membrane-based antibody microarrays by simply pipetting microliter antibody reagents onto pre-patterned and stored membranes, such as nitrocellulose membranes. Current methods of creating 3D microarrays involve expensive lithography or microarray printing technology and are unavailable to most biological/biomedical research labs. The nCLA uses pre-patterned membrane, such as nitrocellulose membrane, micro-spots separated by hydrophobic barriers to allow microarrays to be made as antibodies self-assemble into the micro-spots. The nCLA was prepared with a “selective background de-activation” technique to create reactive micro-spots of nitrocellulose and a de-activated nitrocellulose background. The nCLA demonstrated superior sensitivity compared to both the 2D assays and conventional ELISA while circumventing the necessity for a microarray spotter and using 50 times less sample/reagent volume required by ELISA. After undergoing an 8-month storage period at −20° C., the nCLA exhibited consistent antibody microarray-forming capabilities compared to its initial functionality.

### Example 2

Reverse Compartmentalized Linker Array (rCLA)

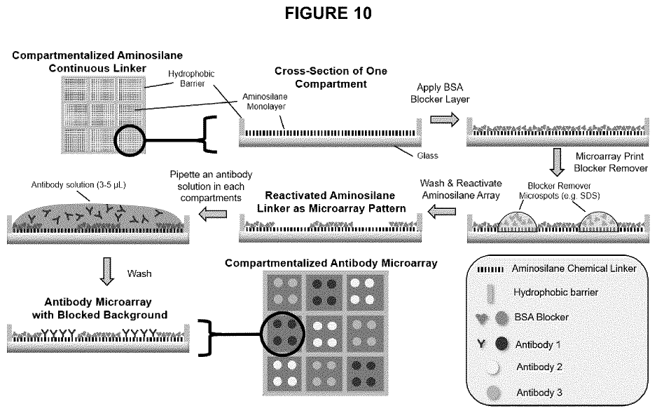

The reverse compartmentalized linker array (rCLA) is a prepatterned microchip designed for creating bio-microarrays by pipetting the bioreagent solution (e.g., antibody, protein, cell) into microliter sized compartments where the bioreagent will self-assemble into the microarray pattern. This self-assembly is facilitated by functionalizing the chip's surface with linker molecules (e.g., aminosilane) and deactivating the background of the microarray while leaving microspots of active chemical linker to adhere pipetted bioreagents. This is done by first deactivating/blocking the entire surface with a blocker protein, such as BSA, followed by microarray printing a chemical to disrupt the aminosilane-bioreagent interaction and remove the BSA blocker molecules from the printed areas. This reverts the microarray spots to un-blocked and reactivate the aminosilane linkers within the micro-spots, which capable of binding bioreagent again. This process can be seen in FIG. 10, using the formation of antibody microarray with aminosilane linker as an example. The compartmentalization of the rCLA is created by applying a hydrophobic barrier in a grid on the chip allowing for separation of many microarrays. Compartmentalization allows users to create many different microarrays on a single chip with reduced risk of cross-reactivity from surrounding assays.

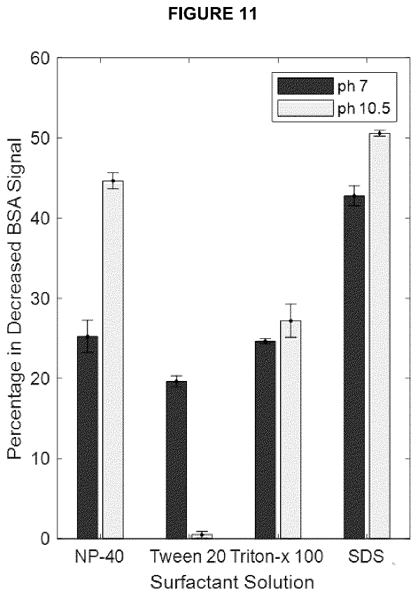

Four surfactants at pH 7 and pH 10.5 were investigated for removing BSA from aminosilane: NP-40, Tween-20, Triton-x 100, and sodium dodecyl sulfate (SDS). The results are shown on FIG. 11. Their effectiveness was determined by their ability to reduce the signal from FITC-tagged BSA (FBSA) compared to an unstripped FBSA-coated aminosilane slide. At pH above 10, the amino group of aminosilane becomes deprotonated losing its positive charge which is one of its mechanisms for protein adhesion [14]. SDS in pH 10.5 removed the most BSA and was selected as the surfactant.

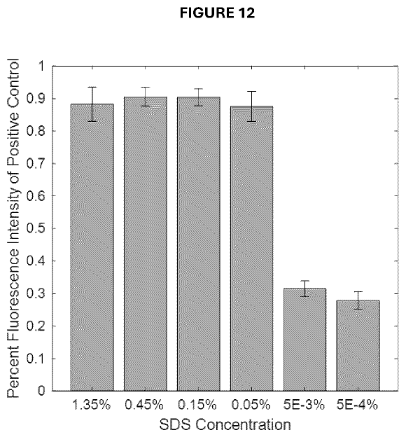

Six concentrations of SDS were compared for their ability to restore protein adhesion abilities to aminosilane after blocking with BSA (FIG. 12). This was measured by the fluorescence intensity of the attached FITC-tagged antibody. The stripped aminosilane was compared with an unaltered aminosilane-coated slide positive control (PC). Concentrations greater than 0.05% SDS all restored about 85% of aminosilane's function for antibody binding. 1.35% to 0.05% were further investigated for their potential as microarray inks.

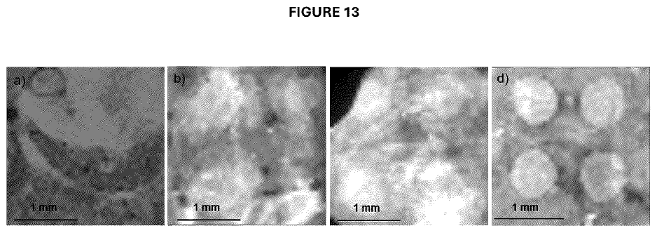

When microarraying the SDS, concentrations of SDS greater than 0.05% also reactivated the background aminosilane as it was washed away resulting in microspots indistinguishable from the background (FIG. 13). Therefore, 0.05% SDS was chosen as the printing concentration for future steps.

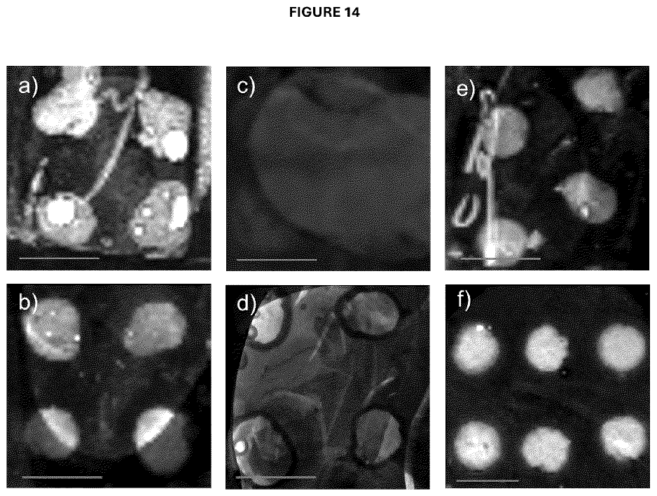

Initial washing methods for removing the microarrayed SDS used a pipetted to slowly apply washing buffer to the surface. Temperatures below 20° C. with SDS solutions can cause the SDS to precipitate from the solution and reduce its efficacy so pipetting wash buffer at 10° C. was tried. This method provided marginal improvement. To reduce the concentration, the slides were placed into a large volume of washing buffer at different insertion orientations, parallel to the buffer surface and perpendicular to the buffer surface. This method was less effective than the pipette. Trying a higher flow rate, a squirt bottle was used to flush the surface of the chip with ~5 mL/s of buffer. This method provided improved results. Washing methods with higher interaction with the spotted SDS seemed to remove the surfactant more effectively before it could interact with the background. Therefore, a stream of ~50 ml/s of DI water was used to remove the SDSs which yielded the microarray in FIG. 14f.

### FULL CITATIONS FOR DOCUMENTS REFERRED TO IN THE APPLICATION

[1] D. Juncker, S. Bergeron, V. Laforte, H. Li, Cross-reactivity in antibody microarrays and multiplexed sandwich assays: shedding light on the dark side of multiplexing, Curr Opin Chem Biol 18 (2014) 29-37. https://doi.org/10.1016/j.cbpa.2013.11.012.
[2] Y. Li, Q. Yang, M. Li, Y. Song, Rate-dependent interface capture beyond the coffee-ring effect, Sci Rep 6 (2016) 24628. https://doi.org/10.1038/srep24628.
[3] R. Aggarwal, B. Ferris, H. Li, Compartmentalized Linker Array: A Scalable and Transferrable Microarray Format for Multiplexed Immunoassays, Anal Chem 95 (2023) 9068-9075. https://doi.org/10.1021/acs.analchem.3c01442.
[4] R. Goodrum, R. T. Aggarwal, H. Li, Gold-nanoparticle-embedded membrane (GEM) for highly sensitive multiplexed sandwich immunoassays, Sens Actuators B Chem 410 (2024) 135731. https://doi.org/10.1016/j.snb.2024.135731.
[5] Giuseppe Cardillo, Four parameters logistic regression—There and back again, (2012). https://github.com/dnafinder/logistic4 (accessed Oct. 31, 2024).
[6] J. Štěpánek, M. Přibyl, D. Šnita, M. Marek, Microfluidic chip for fast bioassays-evaluation of binding parameters, Biomicrofluidics 1 (2007) 024101. https://doi.org/10.1063/1.2723647.
[7] Y.-S. Sun, X. Zhu, Characterization of Bovine Serum Albumin Blocking Efficiency on Epoxy-Functionalized Substrates for Microarray Applications, SLAS Technol 21 (2016) 625-631. https://doi.org/10.1177/2211068215586977.
[8] P. Chen, Y. Qiu, S. Chen, Y. Zhao, Y. Wu, Y. Wang, Insights into the effects of different drying methods on protein oxidation and degradation characteristics of golden pompano (Trachinotus ovatus), Front Nutr 9 (2022). https://doi.org/10.3389/fnut.2022.1063836.
[9] A. Kimble, C. Ratanski, T. A. Kremer, Chemical Changes Over Time Associated with Protein Drying, Biomed Instrum Technol 57 (2023) 52-57. https://doi.org/10.2345/0899-8205-57.2.52.
[10] M. L. Uribe, I. Marrocco, Y. Yarden, EGFR in Cancer: Signaling Mechanisms, Drugs, and Acquired Resistance, Cancers (Basel) 13 (2021) 2748. https://doi.org/10.3390/cancers13112748.
[11] R. Duszkiewicz, J. Strzelczyk, E. Chelmecka, J. K. Strzelczyk, Serum Concentrations of IGF-1R, ERK2, and EGFR and Their Clinical Significance in Patients with Neuroendocrine Tumors, Applied Sciences 14 (2024) 6998. https://doi.org/10.3390/app 14166,998.
[12] S. F. Josephs, T. E. Ichim, S. M. Prince, S. Kesari, F. M. Marincola, A. R. Escobedo, A. Jafri, Unleashing endogenous TNF-alpha as a cancer immunotherapeutic, J Transl Med 16 (2018) 242. https://doi.org/10.1186/s12967-018-1611-7.
[13] L. Conti, S. Gessani, GM-CSF in the generation of dendritic cells from human blood monocyte precursors: Recent advances, Immunobiology 213 (2008) 859-870. https://doi.org/10.1016/j.imbio.2008.07.017.
[14] Issa, A. A., El-Azazy, M., & Luyt, A. S. (2019). Kinetics of alkoxysilanes hydrolysis: An empirical approach. Scientific Reports, 9 (1), 17624. https://doi.org/10.1038/s41598-019-54095-0.

1. A method of preparing a compartmentalized membrane array comprising:
(a) adding one or more barriers on a surface of a membrane to form one or more compartments;
(b) printing a hydrophilic polymer solution in the one or more compartments on the surface of the membrane in a plurality of distinct regions;
(c) adding a solution comprising a blocking reagent to the one or more compartments, wherein the blocking reagent binds to the membrane surface in regions other than the plurality of distinct regions; and
(d) washing the membrane to remove the hydrophilic polymer solution and excess blocking reagent, if present, to form the compartmentalized membrane array.

2. The method of claim 1, wherein the membrane is a nanostructure, hydrogel or a paper membrane.

3. The method of claim 1, wherein the membrane is a cellulose membrane, optionally a nitrocellulose membrane.

4. The method of claim 1, wherein the one or more barriers are hydrophobic barriers, optionally formed on the surface of the membrane with a hydrophobic barrier pen.

5. The method of claim 1, wherein the hydrophilic polymer solution is an aqueous polyethylene glycol (PEG) or a glycerol solution.

6. The method of claim 1, wherein the blocking reagent solution further comprises a buffer and/or the blocking reagent is a protein blocking reagent.

7. The method of claim 6, wherein the blocking reagent is selected from bovine serum albumin (BSA) and succinic anhydride (SA).

8. The method of claim 1, wherein the hydrophilic polymer solution prevents or substantially reduces the blocking reagent from binding to the membrane in the plurality of distinct regions.

9. The method of claim 1, wherein the hydrophilic polymer solution is removed by washing the membrane with a suitable solvent.

10. A compartmentalized membrane array prepared using a method of claim 1.

11. A method of preparing a reverse compartmentalized array comprising
(a) coating a surface of a substrate with linker molecules to form a linker monolayer;
(b) coating the linker monolayer with a blocking reagent solution to form a blocking reagent layer on the linker monolayer;
(c) printing a solution of a blocking reagent remover on a plurality of distinct regions on the blocker reagent layer; and
(d) washing the substrate to remove the blocking reagent remover and the blocking reagent on the plurality of distinct regions to form the reverse compartmentalized array,
wherein the substrate comprises one or more barriers on the surface that form one or more compartments.

12. The method of claim 11, wherein the substrate is selected from silicon, glass, a nanostructure membrane, a hydrogel membrane, a paper membrane, mica, and rigid plastics.

13. The method of claim 11, wherein the substrate is glass or a nitrocellulose membrane.

14. The method of claim 11, wherein the linker molecules are selected from an aminosilane, a poly-L-lysine, an epoxysilane, an aldehyde silane, a trichloro(alkyl) silane, and streptavidin.

15. The method of claim 11, wherein the blocking reagent is a protein blocking reagent present in the blocking reagent solution in an amount of about 0.2% w/v to about 20% w/v, about 0.5% w/v to about 15% w/v or about 10% w/v.

16. The method of claim 15, wherein the blocking reagent is selected from bovine serum albumin (BSA) and succinic anhydride (SA).

17. The method of claim 11, wherein the solution of the blocking reagent remover has a pH of about 10 to about 11 and wherein blocking agent remover is present in the solution of the blocking reagent remover in an amount of about 0.05% w/v to about 3% w/v.

18. The method of claim 11, wherein the one or more barriers are hydrophobic barriers.

19. A reverse compartmentalized array prepared using a method of claim 11.

20. A reverse compartmentalized array comprising:
(a) a substrate comprising a surface coated with a linker molecule monolayer;
(b) a blocking reagent layer on the linker molecule monolayer; and
(c) a plurality of distinct regions in the blocker reagent layer wherein the blocking reagent layer has been removed to expose the linker molecule monolayer;
wherein the substrate comprises one or more barriers on the surface that form one or more compartments.
